(19) United States
(12) Patent Application Publication
(10) Pub. No.: US 2018/0086765 A1
(43) Pub. Date: Mar. 29, 2018

(54) NOVEL INTERMEDIATES FOR PREPARING DPP-IV INHIBITORS, PREPARING METHOD THEREOF AND PREPARING METHOD OF DPP-IV INHIBITORS USING THE SAME

(71) Applicant: KYUNG DONG PHARM. CO., LTD., Gyeonggi-do (KR)

(72) Inventors: Byoung Suk LEE, Seoul (KR); Sang Hoon SHIN, Gyeonggi-do (KR); Yoo Kil AN, Gyeonggi-do (KR); Eun Jeong CHUN, Gyeonggi-do (KR)

(21) Appl. No.: 15/563,403

(22) PCT Filed: Feb. 22, 2016
(86) PCT No.: PCT/KR2016/001716

§ 371 Date: Sep. 29, 2017

(30) Foreign Application Priority Data
Jun. 16, 2015 (KR) 10-2015-0085346

(51) Int. Cl. C07D 487/04; C07C 271/22; C07D 213/64; C07C 269/04; C07D 241/08

(52) U.S. Cl. CPC C07D 487/04; C07C 271/22; C07D 241/08; C07C 269/04; C07D 213/64

(57) ABSTRACT

Disclosed are novel intermediates for use in preparing DPP-IV inhibitors, methods for preparing the same, and methods for preparing DPP-IV inhibitors using the same. Using the novel intermediates of the present invention, highly pure DPP-IV inhibitors can be produced in a simple and economical manner at a high yield.

### TECHNICAL FIELD

The present invention relates to novel intermediates for preparing dipeptidyl peptidase IV (hereinafter referred to as DPP-IV) inhibitors, method for preparing the same, and method for preparing DPP-IV inhibitors using the same.

### BACKGROUND ART

Among hormone candidates for the therapy of diabetes mellitus, except for insulin, is glucagon like peptide-1 (hereinafter referred to as GLP-1), which is a kind of incretin hormones. Particularly, in patients with type 2 diabetes mellitus, when DPP-IV which degrades GLP-1 is inhibited, the level of GLP-1 is elevated, with the consequent reduction of blood sugar levels (Diabetes. 1998, 47(11), 1663-1670). In addition, it is reported that the selective inhibition of DPP-IV prevents the degradation of GLP-1, resulting in promoting insulin secretion (Diabetes. 1998, 47(5), 764-769).

Sitagliptin, the first DPP-IV inhibitor for the treatment of type 2 diabetes mellitus, is disclosed, together with the preparation of sitagliptin hydrochloride through the following Reaction Scheme 1, in WO 2003/004498:

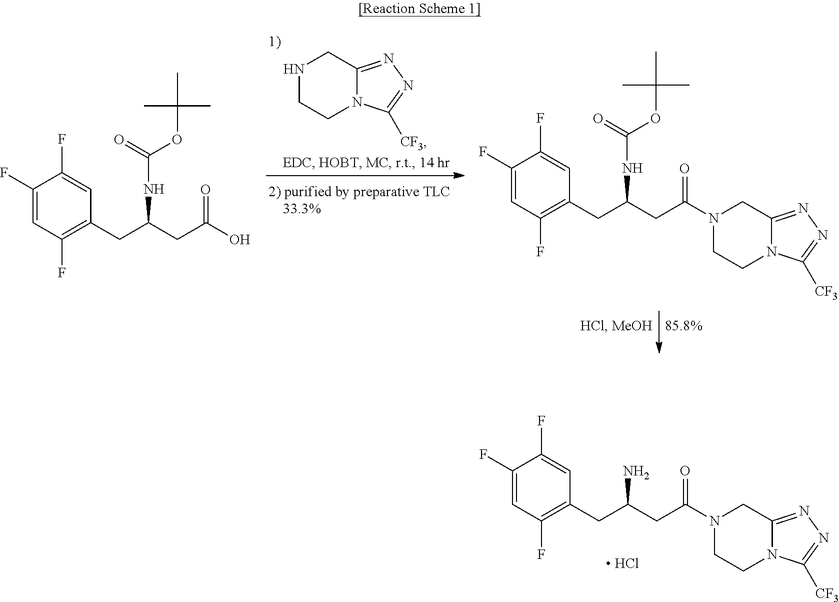 [SMILES: CC(C)(C)OC(=O)N[C@@H](CC(=O)O)Cc1cc(F)c(F)cc1F ; FC(F)(F)c1nnc2n1CCNC2 ; CC(C)(C)OC(=O)N[C@@H](CC(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F ; "· HCl"]

As illustrated in Reaction Scheme 1, (R)-3-(t-butoxycarbonylamino)-4-(2,4,5-trifluorophenyl)butanoic acid is reacted for about 14 hours with 3-(trifluoromethyl)-5,6,7,8-tetrahydro-[1,2,4]triazolo[4,3-α]pyrazine in the presence of 1-ethyl-3-(3-dimethylaminopropyl)carbodiimide (EDC) and hydroxybenzotriazole (HOBT) in dichloromethane to give 7-[(3R)-3-[(1,1-dimethylethoxycarbonyl)amino]-4-(2,4,5-trifluorophenyl)butanoyl]-3-(trifluoromethyl)-5,6,7,8-tetrahydro-1,2,4-triazolo[4,3-α]pyrazine as an intermediate, followed by the treatment of the intermediate with saturated hydrochloric acid in methanol to afford sitagliptin hydrochloride.

However, this reaction scheme employs the very expensive reagents EDC and HOBT. Further, these reagents are difficult to extract as separated layers, and chromatographic purification is not suitable for the industrial production on a mass scale. Moreover, the intermediate is produced at as low yield as 33.3%.

WO 2004/087650 suggests the following Reaction Scheme 2 through which sitagliptin is produced from (3S)-3-hydroxy-4-(2,4,5-trifluorophenyl)butanoic acid in a five-step process:

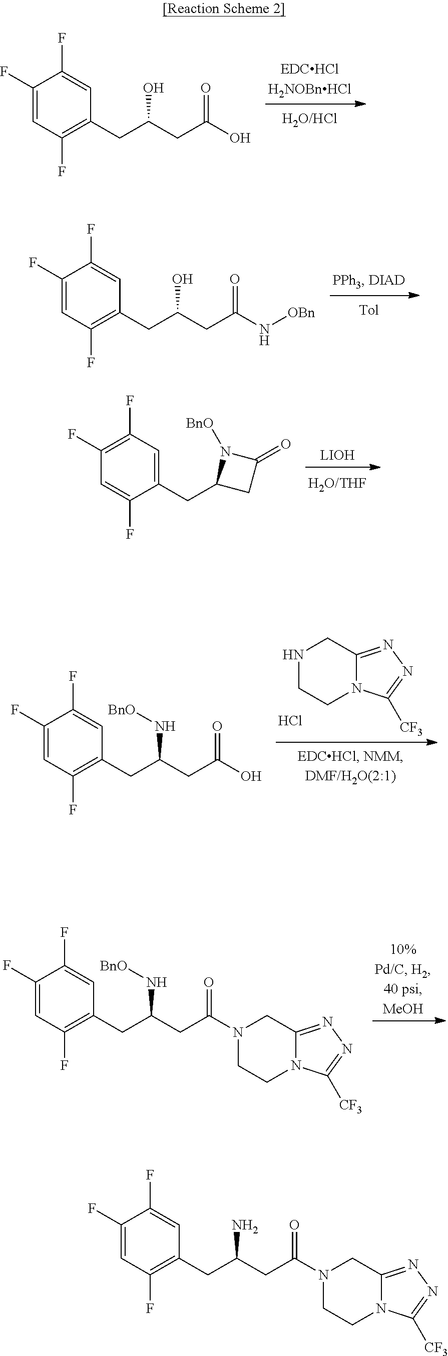

As can be seen in Reaction Scheme 2, 1-ethyl-3-(3-dimethylaminopropyl)carbodiimide (EDC) is used in the first step and used again for reaction with 3-(trifluoromethyl)-5,6,7,8-tetrahydro-[1,2,4]triazolo[4,3-α]pyrazine hydrochloride in fourth step. However, EDC is difficult to store and handle with as it generally requires a condition of −20° C. for its storage. In addition, since this strategy employs hydrogenation in the presence of palladium/carbon for deprotecting the amino-protecting benzyloxy group, the expensive metal catalyst causes an increase in production cost while hydrogen gas is at the risk of explosion, both of which acts as a barrier to the industrialization of the process.

WO 2009/064476 describes the production of sitagliptin via the route given in the following Reaction Scheme 3.

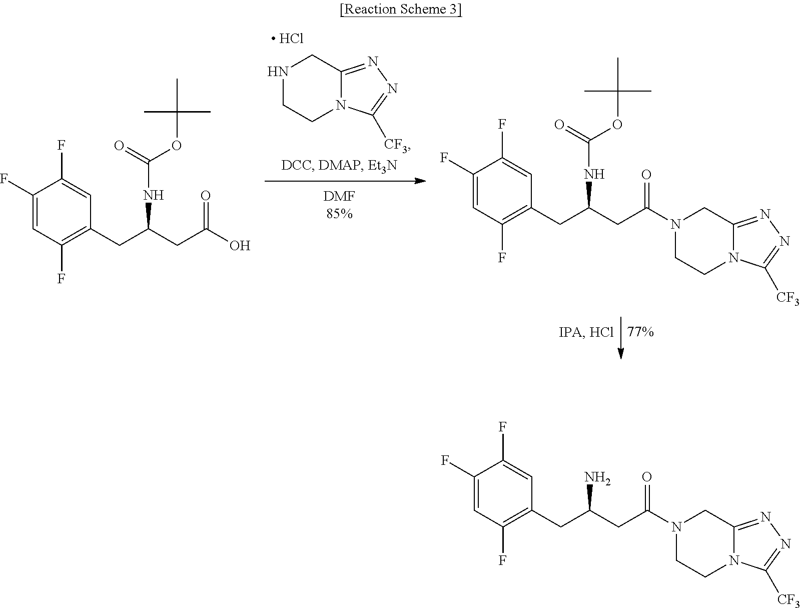 [SMILES: "· HCl" ; CC(C)(C)OC(=O)N[C@@H](CC(=O)O)Cc1cc(F)c(F)cc1F ; FC(F)(F)c1nnc2n1CCNC2 ; CC(C)(C)OC(=O)N[C@@H](CC(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F]

As illustrated in Reaction Scheme 3, (R)-3-(t-butoxycarbonylamino)-4-(2,4,5-trifluorophenyl)butanoic acid is reacted with 3-(trifluoromethyl)-5,6,7,8-tetrahydro-[1,2,4]triazolo[4,3-α]pyrazine hydrochloride in the presence of N,N′-dicyclohexylcarbodiimide (DCC), 4-dimethylaminopyridine(DMAP), and triethylamine in dimethylformamide for as long a long period of time as one or more days to give 7-[(3R)-3-[(1,1-dimethylethoxycarbonyl)amino]-4-(2,4,5-trifluorophenyl)butanoyl]-3-(trifluoromethyl)-5,6,7,8-tetrahydro-1,2,4-triazolo[4,3-α]pyrazine, followed by treating this intermediate with hydrochloric acid in 2-propanol to afford sitagliptin.

After completion of the reaction, however, a lot of by-products is produced due to the use of DCC and DMAP, which additionally requires a filtration process for removing the by-products. Further, dimethylformamide, having a boiling point of as high as about 152° C. is used in an excess amount 12.5 times the weight of (R)-3-(t-butoxycarbonylamino)-4-(2,4,5-trifluorophenyl)butanoic acid, which renders layer separation difficult in subsequent concentration and extraction processes, resulting in a decrease in the purity of the product.

To solve the problems encountered in conventional techniques, the present inventors have found a novel intermediate of sitagliptin and a preparation method thereof with which highly pure sitagliptin can be produced simply and economically in a mild condition at high yield and which can be applied to industrialization.

In addition, the DPP-IV inhibitor evogliptin was first disclosed in Korean Patent Publication No. 2008-0094604 in which the following Reaction Scheme 4 is also suggested as a route for preparing evogliptin.

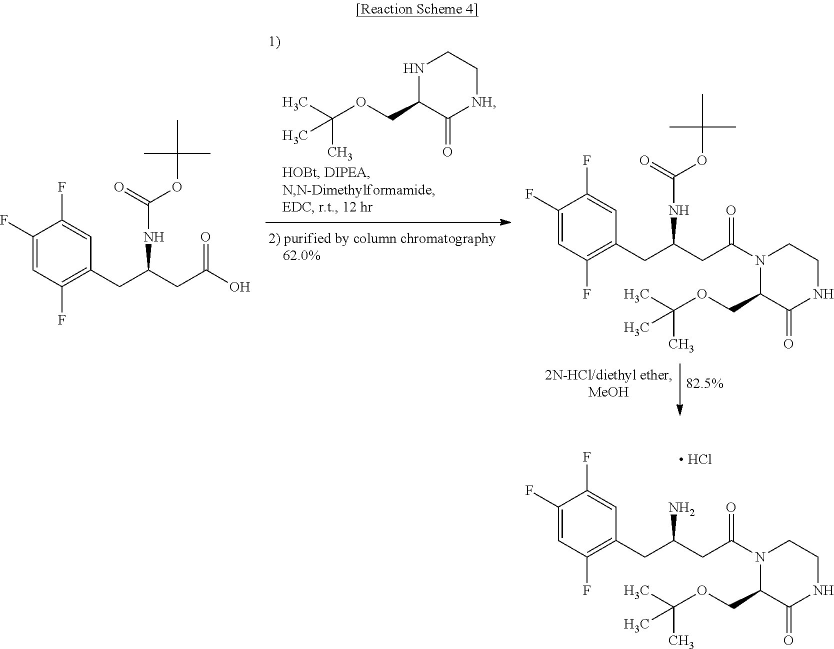 [SMILES: CC(C)(C)OC(=O)N[C@@H](CC(=O)O)Cc1cc(F)c(F)cc1F ; CC(C)(C)OC[C@H]1NCCNC1=O ; CC(C)(C)OC[C@@H]1C(=O)NCCN1C(=O)C[C@@H](Cc1cc(F)c(F)cc1F)NC(=O)OC(C)(C)C ; CC(C)(C)OC[C@@H]1C(=O)NCCN1C(=O)C[C@H](N)Cc1cc(F)c(F)cc1F ; "· HCl"]

As shown in Reaction Scheme 4, (R)-3-(t-butoxycarbonylamino)-4-(2,4,5-trifluorophenyl)butanoic acid is reacted with (R)-(3-t-butoxymethyl)piperazin-2-one in the presence of 1-ethyl-3-(3-dimethylaminopropyl)carbodiimide (EDC), 1-hydroxybenzotriazole (HOBT), and the tertiary amine diisopropylethylamine (DIPEA) in N,N-dimethylformamide for about 12 hours to prepare t-butyl (R)-4-[(R)-2-(t-butoxymethyl)-3-oxopiperazin-1-yl]-4-oxo-1-(2,4,5-trifluorophenyl)butan-2-ylcarbamate, an intermediate of evogliptin, which is then dissolved in methanol and treated with 2N—HCl/diethylether to afford evogliptin hydrochloride.

However, EDC and HOBT, used in this reaction scheme, are very expensive reagent. Further, these reagents are difficult to extract as separated layers, and column chromatographic purification is not suitable for the industrial production on a mass scale. Moreover, the intermediate is produced at as low yield as 62.0%.

Korean Patent Publication No. 2010-0109493 discloses the production of evogliptin via the following Reaction Scheme 5.

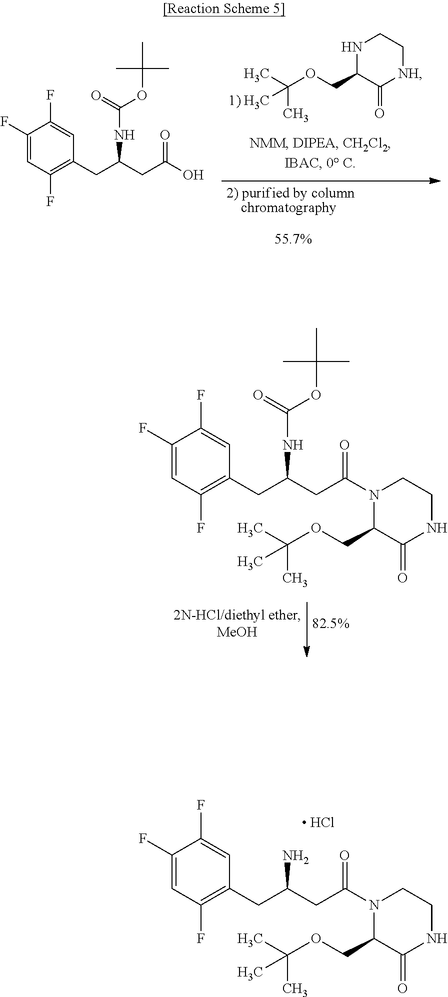

As shown in Reaction Scheme 5, (R)-3-(t-butoxycarbonylamino)-4-(2,4,5-trifluorophenyl)butanoic acid is reacted at 0° C. with (R)-(3-t-butoxymethyl)piperazin-2-one in the presence of isobutylchloroformate (IBCF), 4-methylmorpholine (NMM), and the base diisopropylethylamine (DIPEA) in dichloromethane to prepare t-butyl (R)-4-[(R)-2-(t-butoxymethyl)-3-oxopiperazin-1-yl]-4-oxo-1-(2,4,5-trifluorophenyl)butan-2-ylcarbamate, an intermediate of evogliptin, which is then dissolved in methanol and treated with 2N—HCl/diethylether to afford evogliptin hydrochloride.

However, IBCF used in the reaction is difficult to store and handle with because it is decomposed at wet condition and highly sensitive to moisture and thus requires cold storage. Further, column chromatographic purification is not suitable for the industrial production on a mass scale. Moreover, the intermediate is produced at as low yield as 55.7%.

Korean Patent Publication No. 2010-0109494 introduces the production of evogliptin through the following Reaction Scheme 6.

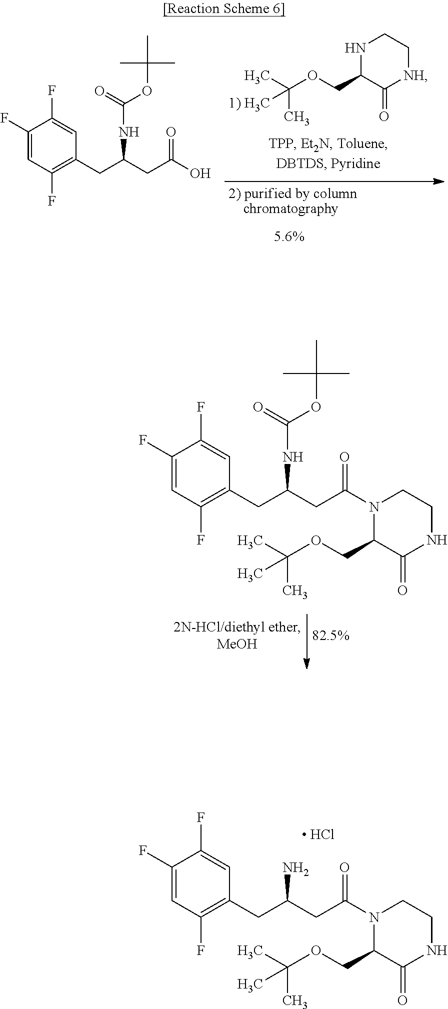

As shown in Reaction Scheme 6, (R)-3-(t-butoxycarbonylamino)-4-(2,4,5-trifluorophenyl)butanoic acid is reacted with (R)-(3-t-butoxymethyl)piperazin-2-one in the presence of bis(2,2′-benzothiazolyl)disulfide (DBTDS), and the bases triphenylphosphine (TPP), triethylamine and pyridine in toluene to prepare t-butyl (R)-4-[(R)-2-(t-butoxymethyl)-3-oxopiperazin-1-yl]-4-oxo-1-(2,4,5-trifluorophenyl)butan-2-ylcarbamate, an intermediate of evogliptin, which is then dissolved in methanol and treated with 2N—HCl/diethylether to afford evogliptin hydrochloride.

After completion of the reaction, however, quantities of 2-benzothiazolthiol (MBT) and triphenylphosphine oxide are produced as by-products, which is a cause of reducing the purity and yield of the product of interest. Further, purification by column chromatography is not suitable for the industrial production on a mass scale. Moreover, the intermediate is produced at as low yield as 5.6%, which seems to be attributed to the column chromatographic purification.

In order to solve the problems encountered in the prior arts, the present inventors have found a novel intermediate of evogliptin, and a preparing method thereof, which can be applied to industrialization of the product of interest.

In addition, retagliptin, a DPP-IV inhibitor, was first disclosed, together with the following Reaction Scheme 7 as the production route thereof, in Korean Patent Publication No. 2011-0002003.

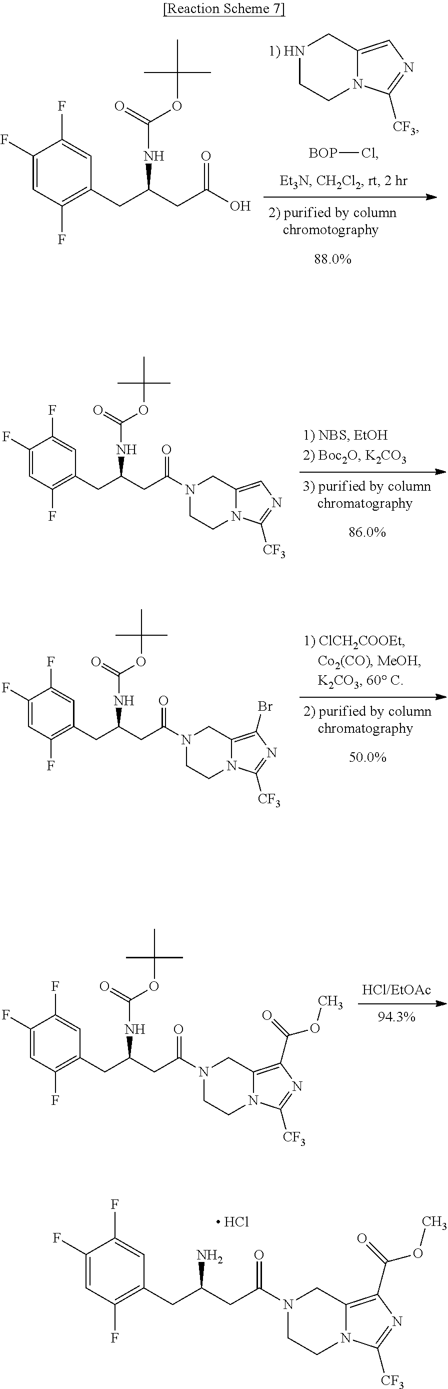

As illustrated in Reaction Scheme 7, (R)-3-(t-butoxycarbonylamino)-4-(2,4,5-trifluorophenyl)butanoic acid is reacted with 3-trifluoromethyl-5,6,7,8-tetrahydro-imidazo[1,5-a] pyrazine in the presence of bis(2-oxo-3-oxazolidinyl)phosphonic acid chloride (BOP-Cl) and triethylamine in dichloromethane to give (R)-[3-oxo-1-(2,4,5-trifluoro-benzyl)-3-(3-trifluoromethyl-5,6-dihydro-8H-imidazo[1,5-α]pyrazin-7-yl)-propyl]-carbamic acid t-butyl ester, an intermediate of retagliptin, which is then subjected to substitution with bromine and methylester, followed by the removal of the amine protecting group to afford retagliptin hydrochloride.

However, the above preparing method is difficult to apply to the industrial production on a mass scale not only because many processes are needed, but also because purification by column chromatography is conducted in most of the processes. The intermediate is synthesized at yield as low as 50.0%.

An alternative route of producing retagliptin is introduced as the following Reaction Scheme 8. As shown, condensation is conducted in the presence of the condensing agent bis(2-oxo-3-oxazolidinyl)phosphonic acid chloride (BOP-Cl), followed by deprotecting the amino-protecting group in the presence of an acid to afford retagliptin. However, this route is described with neither examples nor production yields given thereto.

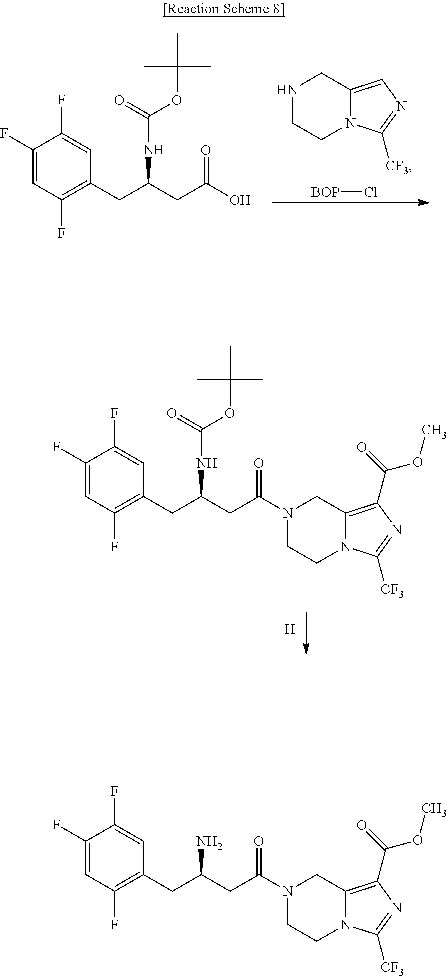

In order to solve the problems encountered in the prior arts, the present inventors have found a novel intermediate of retagliptin, and a preparing method thereof, which can be applied to industrialization of the product of interest.

### DISCLOSURE OF INVENTION

### Technical Problem

It is an object of the present invention to provide a novel intermediate for use in preparing a DPP-IV inhibitor.

It is another object of the present invention to provide a method for preparing the novel intermediate.

It is a further object of the present invention to provide a method for preparing a DPP-IV inhibitor with high purity in a simple manner at high yield using the novel intermediate.

### Solution to Problem

In accordance with an aspect thereof, the present invention provides novel intermediates for use in preparing DPP-IV inhibitors.

In detail, the novel intermediates of the present invention are represented by the following Chemical Formula 1:

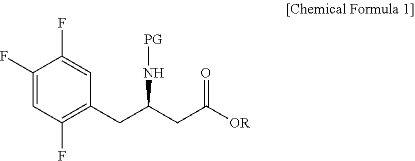

wherein,

R is pentafluorophenyl, 4-nitrophenyl, or 2-pyridyl, and

PG is an amine protecting group.

In accordance with another aspect thereof, the present invention provides a method for preparing a compound represented by Chemical Formula 1.

In an exemplary embodiment, the method for preparing the novel intermediate represented by Chemical Formula 1 comprises reacting a compound represented by the following Chemical Formula 2 with a compound represented by the following Chemical Formula 3 in the presence of a base:

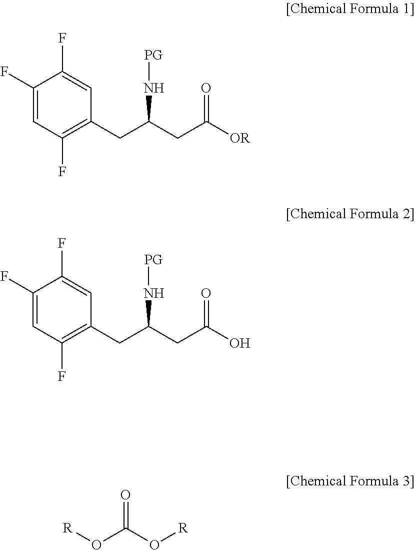

wherein,

R is pentafluorophenyl, 4-nitrophenyl, or 2-pyridyl, and

PG is an amine protecting group.

In accordance with a further aspect thereof, the present invention provides methods for preparing DPP-IV inhibitors, using novel intermediates represented by the following Chemical Formula 1.

In detail, the method comprises: (S1) reacting a compound represented by the following Chemical Formula 2 with a compound represented by the following Chemical Formula 3 in the presence of a base to prepare the novel intermediate represented by the following Chemical Formula 1; and (S2) reacting the compound represented by the Chemical Formula 1 with any one of compounds represented by the following Chemical Formulas 4a to 4c or a salt thereof to afford any one of compounds represented by the following Chemical Formulas 5a to 5c:

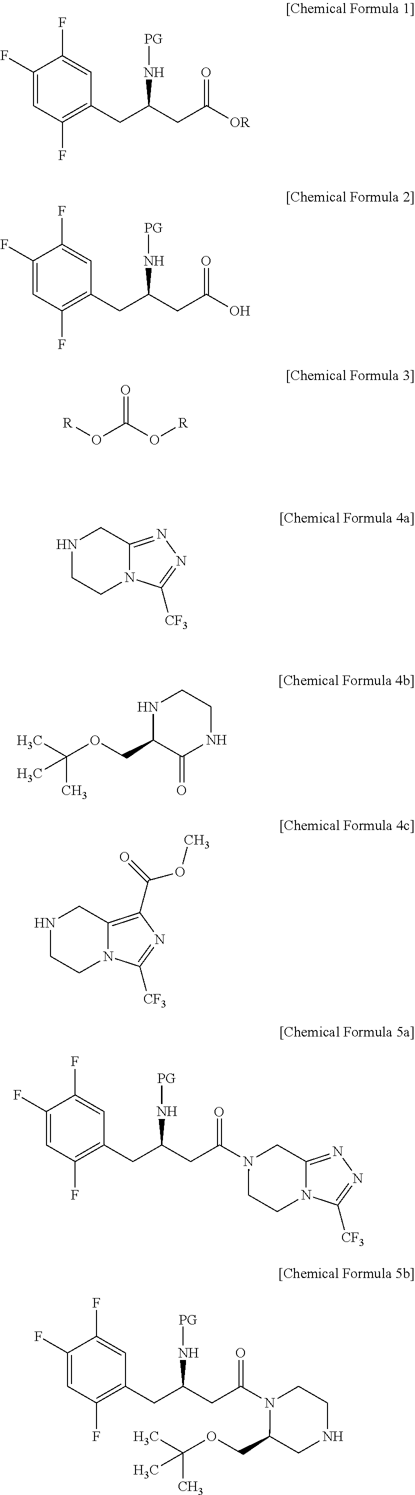

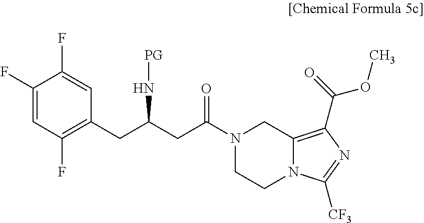

wherein,

R is pentafluorophenyl, 4-nitrophenyl, or 2-pyridyl, and

PG is an amine protecting group.

Further, the method for preparing DPP-IV inhibitors may comprise (S3) removing the amine protecting group to synthesize any one of compounds represented by the following Chemical Formula 6a (sitagliptin), Chemical Formula 6b (evogliptin) and Chemical Formula 6c (retagliptin):

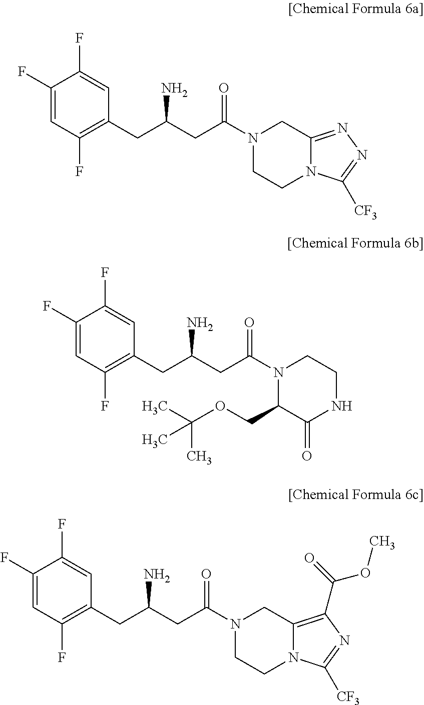 [SMILES: N[C@@H](CC(=O)N1CCn2c(nnc2C(F)(F)F)C1)Cc1cc(F)c(F)cc1F ; CC(C)(C)OC[C@@H]1C(=O)NCCN1C(=O)C[C@H](N)Cc1cc(F)c(F)cc1F ; COC(=O)c1nc(C(F)(F)F)n2c1CN(C(=O)C[C@H](N)Cc1cc(F)c(F)cc1F)CC2]

### Advantageous Effects of Invention

A highly pure DPP-IV inhibitor can be produced in a simple and economical manner at high yield, using the novel intermediate of the present invention.

### MODE FOR THE INVENTION

An aspect of the present invention addresses novel intermediates for use in preparing DPP-IV inhibitors, and methods for preparing the same.

Also, contemplated in accordance with another aspect of the present invention are methods for preparing DPP-IV inhibitors, using the novel intermediates.

These and other features will be described in detail, below.

Novel Intermediates

The novel intermediate of the present invention is a compound represented by the following Chemical Formula 1:

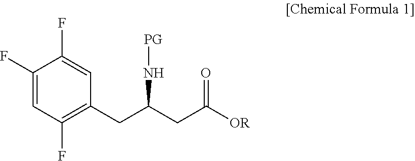

wherein,

R is pentafluorophenyl, 4-nitrophenyl, or 2-pyridyl, and

PG is an amine protecting group.

In the present invention, so long as it is typically used in the art, any amine protecting group may be used as the PG. Examples of the amine protecting group include Boc (t-butyloxycarbonyl), Cbz (benzyloxycarbonyl), Fmoc (fluorenylmethyloxycarbonyl), acetyl or benzoyl, but are not limited thereto.

In accordance with an embodiment of the present invention, the compound represented by Chemical Formula 1 may be (R)-pentafluorophenyl 3-(t-butoxycarbonylamino)-4-(2,4,5-trifluorophenyl)butanoate represented by the following Chemical Formula 1a.

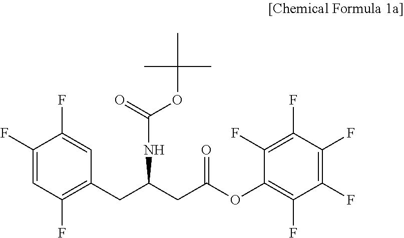 [SMILES: CC(C)(C)OC(=O)N[C@@H](CC(=O)Oc1c(F)c(F)c(F)c(F)c1F)Cc1cc(F)c(F)cc1F]

In accordance with another exemplary embodiment of the present invention, the compound represented by Chemical Formula 1 may be (R)-4-nitrophenyl 3-(t-butoxycarbonylamino)-4-(2,4,5-trifluorophenyl)butanoate represented by the following Chemical Formula 1b.

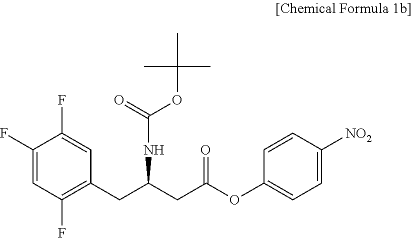 [SMILES: CC(C)(C)OC(=O)N[C@@H](CC(=O)Oc1ccc([N+](=O)[O-])cc1)Cc1cc(F)c(F)cc1F]

In accordance with a further exemplary embodiment of the present invention, the compound represented by Chemical Formula 1 may be (R)-pyridin-2-yl 3-(t-butoxycarbonylamino)-4-(2,4,5-trifluorophenyl)butanoate represented by the following Chemical Formula 1c.

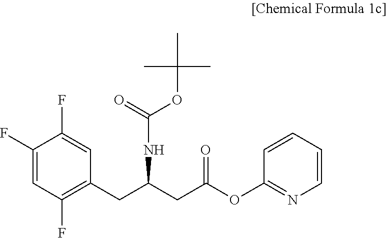 [SMILES: CC(C)(C)OC(=O)N[C@@H](CC(=O)Oc1ccccn1)Cc1cc(F)c(F)cc1F]

The compound represented by Chemical Formula 1 is used as an intermediate for use in preparing DPP-IV inhibitors, particularly, sitagliptin, evogliptin, or retagliptin. By way of the novel intermediates of the present invention, DPP-IV inhibitors with high purity can be produced at high yield.

Preparation Method of Novel Intermediates of Chemical Formula 1

The method for preparing the novel intermediate of Chemical Formula 1 comprises reacting a compound represented by the following Chemical Formula 2 with a compound represented by the following Chemical Formula 3 in the presence of a base:

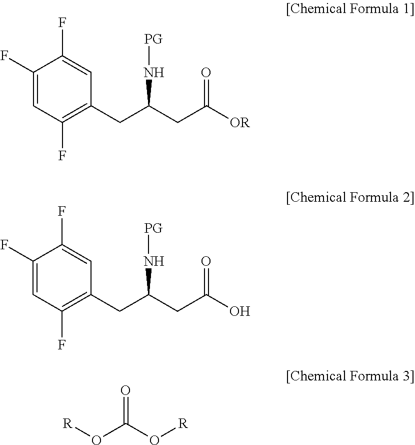

wherein,

R is pentafluorophenyl, 4-nitrophenyl, or 2-pyridyl, and

PG is an amine protecting group.

The amine protecting group is same as described above.

In the present invention, the carbonate derivative of Chemical Formula 3 may preferably comprise phenyl or pyridyl having electron withdrawing group(s). More preferably, the carbonate derivative may be bis(pentafluorophenyl)carbonate, bis(4-nitrophenyl)carbonate, or di-2-pyridyl carbonate.

In the present invention, the carbonate derivative of Chemical Formula 3 is preferably used at a ratio of 1 to 3 molar equivalents to 1 molar equivalent of the compound of Chemical Formula 2, and more preferably at a ratio of 1 to 1.5 molar equivalents.

In the present invention, the base may be preferably selected from the group consisting of sodium carbonate, potassium carbonate, sodium hydroxide, potassium hydroxide, sodium bicarbonate, potassium bicarbonate, triethylamine, trimethylamine, pyridine, N-methylmorpholine, triisopropylamine and diisopropylethylamine. More preferably, the base is triethylamine. Further, the base is preferably used at a ratio of 1 to 3 molar equivalents to 1 molar equivalent of the compound of Chemical Formula 2, and more preferably at a ratio of 1 to 1.5 molar equivalents.

In the present invention, the reaction may be conducted in an organic solvent. The organic solvent may preferably be selected from the group consisting of 2-propanol, acetonitrile, ethylacetate, acetone, tetrahydrofuran, toluene, dichloromethane, dimethylacetamide, dimethylsulfoxide, dimethylformamide and a combination thereof. More preferably, the organic solvent is dimethylformamide. In addition, the organic solvent is preferably used in an amount of 2 to 20 volumes of the compound of Chemical Formula 2, and more preferably in an amount of 3 to 10 volumes.

In the present invention, the reaction may be conducted at a temperature of 0 to 100° C., preferably at a temperature of 0 to 80° C., and more preferably at a temperature of 20 to 70° C.

Preparation of DPP-IV Inhibitors by Way of the Novel Intermediates

In accordance with a further aspect thereof, the present invention addresses methods for preparing DPP-IV inhibitors, using the novel intermediates represented by Chemical Formula 1.

In detail, the method comprises: (S1) reacting a compound represented by the following Chemical Formula 2 with a compound represented by the following Chemical Formula 3 in the presence of a base to prepare a novel intermediate represented by the following Chemical Formula 1; and (S2) reacting the compound represented by the Chemical Formula 1 with any one of compounds represented by the following Chemical Formulas 4a to 4c or a salt thereof to afford any one of compounds represented by the following Chemical Formulas 5a to 5c:

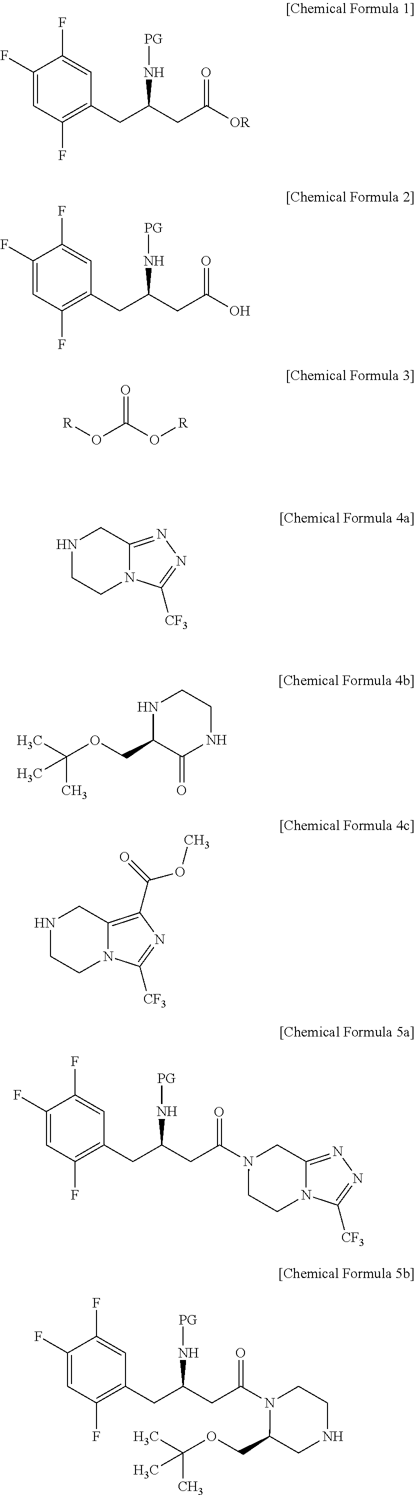

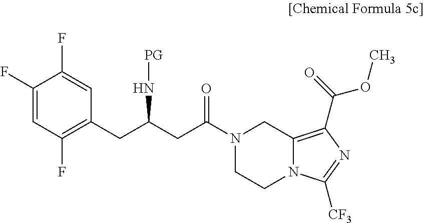

wherein,

R is pentafluorophenyl, 4-nitrophenyl, or 2-pyridyl; and

PG is an amine protecting group.

The amine protecting group is same as described above.

In the present invention, as far as the step (S1) is concerned, reference may be made to the description on the preparation method of the novel intermediates.

In the present invention, the compound represented by Chemical Formula 1 obtained in the step (S1) may be used in the reaction of step (S2) without the isolation thereof.

In the present invention, any one of compounds represented by Chemical Formulas 4a to 4c or a salt thereof is preferably used in step (S2) at a ratio of 1 to 3 molar equivalents to 1 molar equivalent of the compound of Chemical Formula 2 of the step (S1), and more preferably at a ratio of 1 to 1.5 molar equivalents.

In accordance with another exemplary embodiment of the present invention, the reaction of step (S2) may be performed at a temperature of 0 to 100° C., preferably at a temperature of 0 to 80° C. and more preferably at a temperature of 20 to 70° C.

In accordance with another exemplary embodiment of the present invention, the method may further comprise (S3) removing the amine protecting group to afford a compound represented by the following Chemical Formula 6a (sitagliptin), Chemical Formula 6b (evogliptin), or Chemical Formula 6c (retagliptin).

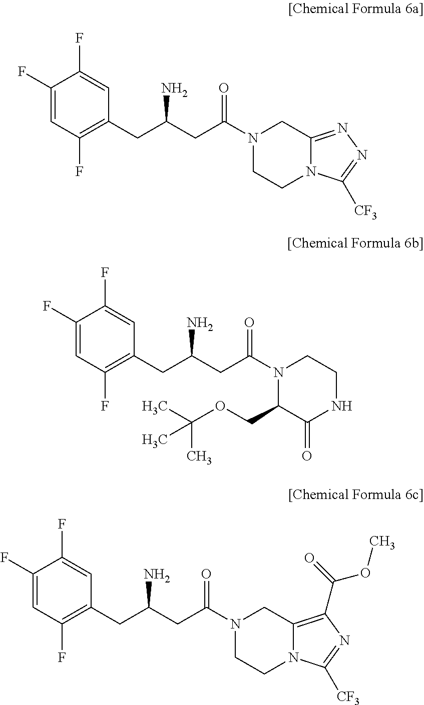 [SMILES: N[C@@H](CC(=O)N1CCn2c(nnc2C(F)(F)F)C1)Cc1cc(F)c(F)cc1F ; CC(C)(C)OC[C@@H]1C(=O)NCCN1C(=O)C[C@H](N)Cc1cc(F)c(F)cc1F ; COC(=O)c1nc(C(F)(F)F)n2c1CN(C(=O)C[C@H](N)Cc1cc(F)c(F)cc1F)CC2]

In the present invention, the step (S3) of removing the amine protecting group may be carried out in a typical deprotecting condition.

Characterized by using the intermediate represented by Chemical Formula 1, the method of the present invention has the advantage of the advantage of preparing a highly pure DPP-IV inhibitor at high yield with a simple procedure. Therefore, the novel intermediate represented by Chemical Formula 1 can be useful for producing DPP-IV inhibitors, particularly, sitagliptin, evogliptin, retagliptin or pharmaceutically acceptable salts thereof on mass scales.

A better understanding of the present invention may be obtained through the following examples that are set forth to illustrate, but are not to be construed as limiting the present invention.

### Example 1: Preparation of (R)-Pentafluorophenyl 3-(t-butoxycarbonylamino)-4-(2,4,5-trifluorophenyl)butanoate (Chemical Formula 1a)

To 100 ml of dimethylformamide was added 33.3 g (0.10 mole) of (R)-3-(t-butoxycarbonylamino)-4-(2,4,5-trifluorophenyl)butanoic acid, and the solution was stirred at 25° C. for 20 min. The solution was mixed with 16.7 ml (0.12 mole) of triethylamine, and stirred for 20 min. To the reaction solution was added 39.4 g (0.10 mole) of bis(pentafluorophenyl)carbonate, and the suspension was stirred at 25° C. for 2 hrs. After completion of the reaction as monitored by TLC, 165 ml of 2-propanol and 330 ml of water were added to the resulting reaction solution, followed by stirring at room temperature for 2 hrs or longer. The precipitated solid was filtered under reduced pressure at room temperature, and the filtrate was washed and dried to afford 46.1 g (92.3%) of (R)-pentafluorophenyl 3-(t-butoxycarbonylamino)-4-(2,4,5-trifluorophenyl)butanoate as a solid.

¹H NMR (CDCl₃, 400 MHz): δ 1.41 (s, 9H), 2.95-2.96 (m, 4H), 4.25-4.29 (m, 1H), 4.91-4.92 (m, 1H), 6.92-7.09 (m, 2H)

Elemental analysis for C₂₁H₁₇F₈NO₄

Calculated—C: 50.5, H: 3.4, N: 2.8

Measured—C: 50.8, H: 3.5, N: 2.8

m.p.: 129˜131° C.

### Example 2: Preparation of (R)-4-nitrophenyl 3-(t-butoxycarbonylamino)-4-(2,4,5-trifluorophenyl)butanoate (Chemical Formula 1b)

To 100 ml of dimethylformamide was added 33.3 g (0.10 mole) of (R)-3-(t-butoxycarbonylamino)-4-(2,4,5-trifluorophenyl)butanoic acid, and the solution was stirred at 25° C. for 20 min. The solution was mixed with 16.7 ml (0.12 mole) of triethylamine, and stirred for 20 min. To the reaction solution was added 30.4 g (0.10 mole) of bis(4-nitrophenyl) carbonate, and the suspension was stirred at 70° C. for 4 hrs. After completion of the reaction as monitored by TLC, 100 ml of 2-propanol and 330 ml of water were added to the resulting reaction solution, followed by stirring at room temperature for 2 hrs or longer. The precipitated solid was filtered under reduced pressure at room temperature, and the filtrate was washed and dried to afford 41.7 g (91.9%) of (R)-4-nitrophenyl 3-(t-butoxycarbonylamino)-4-(2,4,5-trifluorophenyl)butanoate as a solid.

¹H NMR (CDCl₃, 400 MHz): δ 1.39 (s, 9H), 2.81-2.96 (m, 4H), 4.32 (m, 1H), 5.04 (m, 1H), 6.94-8.26 (m, 6H)

Elemental analysis for C₂₁H₂₁F₃N₂O₆

Calculated—C: 55.8, H: 4.7, N: 6.2

Measured—C: 55.8, H: 4.8, N: 6.1

m.p.: 138˜140° C.

### Example 3: Preparation of (R)-pyridin-2-yl 3-(t-butoxycarbonylamino)-4-(2,4,5-trifluorophenyl)butanoate (Chemical Formula 1c)

To 100 ml of dimethylformamide was added 33.3 g (0.10 mole) of (R)-3-(t-butoxycarbonylamino)-4-(2,4,5-trifluorophenyl)butanoic acid, and the solution was stirred at 25° C. for 20 min. The solution was mixed with 16.7 ml (0.12 mole) of triethylamine, and stirred for 20 min. To the reaction solution was added 21.6 g (0.10 mole) of di-2-pyridyl carbonate, and the suspension was stirred at 70° C. for 2 hrs. After completion of the reaction as monitored by TLC, 33 ml of 2-propanol and 330 ml of water were added to the resulting reaction solution, followed by stirring at room temperature for 2 hrs or longer. The precipitated solid was filtered under reduced pressure at room temperature, and the filtrate was washed and dried to afford 37.5 g (91.4%) of (R)-pyridin-2-yl 3-(t-butoxycarbonylamino)-4-(2,4,5-trifluorophenyl)butanoate as a solid.

¹H NMR (CDCl₃, 400 MHz): δ 1.38 (s, 9H), 2.81-2.92 (m, 4H), 4.27-4.28 (m, 1H), 5.14-5.16 (m, 1H), 6.90-8.41 (m, 6H)

Elemental analysis for C₂₀H₂₁F₃N₂O₄

Calculated—C: 58.5, H: 5.2, N: 6.8

Measured—C: 58.5, H: 5.3, N: 6.9

m.p.: 134˜137° C.

### Example 4: Preparation of (R)-t-Butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate (Chemical Formula 5a)

To 100 ml of dimethylformamide was added 33.3 g (0.10 mole) of (R)-3-(t-butoxycarbonylamino)-4-(2,4,5-trifluorophenyl)butanoic acid, and the solution was stirred at 25° C. for 20 min. The solution was mixed with 16.7 ml (0.12 mole) of triethylamine, and stirred for 20 min. To the reaction solution was added 43.3 g (0.11 mole) of bis(pentafluorophenyl)carbonate, and the suspension was stirred at 25° C. for 3 hrs.

After completion of the reaction as monitored by TLC, 25.1 g (0.11 mol) of 3-(trifluoromethyl)-5,6,7,8-tetrahydro-[1,2,4]triazolo[4,3-α]pyrazine hydrochloride was added to the resulting solution, followed by stirring at 70° C. for 2. After completion of the reaction as monitored by TLC, 165 ml of 2-propanol and 330 ml of water were added to the reaction solution, and stirred at room temperature for 2 hrs or longer. The precipitated solid was filtered under reduced pressure at room temperature. To the filtrate was added 165 ml of 2-propanol, and the solution was stirred under reflux for 2 hrs or longer. The resulting reaction mixture was slowly cooled to 10° C. or less, filtered under reduced pressure, washed, and dried to afford 46.1 g (91.3%) of (R)-t-butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate as a solid.

HPLC content: 99.3%

¹H NMR (400 MHz, CDCl₃): δ 1.35 (s, 9H), 3.00 (m, 2H), 3.30 (m, 2H), 3.93 (m, 1H), 4.04-4.24 (m, 2H), 4.23 (s, 1H), 4.35 (m, 1H), 4.97-5.48 (m, 2H), 7.22 (m, 1H), 7.44 (m, 1H), 8.04 (m, 1H)

### Example 5: Preparation of: (R)-t-Butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate (Chemical Formula 5a)

To 100 ml of dimethylformamide was added 33.3 g (0.10 mole) of (R)-3-(t-butoxycarbonylamino)-4-(2,4,5-trifluorophenyl)butanoic acid, and the solution was stirred at 25° C. for 20 min. The solution was mixed with 16.7 ml (0.12 mole) of triethylamine, and stirred for 20 min. To the reaction solution was added 30.4 g (0.10 mole) of bis(4-nitrophenyl)carbonate, and the suspension was stirred at 70° C. for 4 hrs.

After completion of the reaction as monitored by TLC, 25.1 g (0.11 mole of 3-(trifluoromethyl)-5,6,7,8-tetrahydro-[1,2,4]triazolo[4,3-α]pyrazine hydrochloride was added to the resulting solution, followed by stirring at 70° C. for 8 hrs. After completion of the reaction as monitored by TLC, 100 ml of 2-propanol and 330 ml of water were added to the reaction solution, and stirred at room temperature for 2 hrs or longer. The precipitated solid was filtered under reduced pressure. To the filtrate was added 100 ml of 2-propanol, and the solution was stirred under reflux for 2 hrs or longer. The resulting reaction mixture was slowly cooled to 10° C. or less, filtered in a vacuum, washed, and dried to afford 46.5 g (92.0%) of (R)-t-butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate as a solid.

HPLC content: 99.3%

Here, the same spectrum data as in Example 4 was obtained.

### Example 6: Preparation of (R)-t-Butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate (Chemical Formula 5a)

To 100 ml of dimethylformamide was added 33.3 g (0.10 mole) of (R)-3-(t-butoxycarbonylamino)-4-(2,4,5-trifluorophenyl)butanoic acid, and the solution was stirred at 25° C. for 20 min. The solution was mixed with 16.7 ml (0.12 mole) of triethylamine, and stirred for 20 min. To the reaction solution was added 21.6 g (0.10 mole) of di-2-pyridyl carbonate, and the suspension was stirred at 70° C. for 2 hrs.

After completion of the reaction as monitored by TLC, 25.1 g (0.11 mole) of 3-(trifluoromethyl)-5,6,7,8-tetrahydro-[1,2,4]triazolo[4,3-α]pyrazine hydrochloride was added to the resulting solution, followed by stiffing at 70° C. for 2 hrs. After completion of the reaction as monitored by TLC, 33 ml of 2-propanol and 330 ml of water were added to the reaction solution, and stirred at room temperature for 2 hrs or longer. The precipitated solid was filtered under reduced pressure. To the filtrate was added 100 ml of 2-propanol, and the solution was stirred under reflux for 2 hrs or longer. The resulting reaction mixture was slowly cooled to 10° C. or less, vacuum filtered, washed, and dried to afford 45.9 g (90.9%) of (R)-t-butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate as a solid.

HPLC content: 99.2%

Here, the same spectrum data as in Example 4 was obtained.

### Example 7: Preparation of 7-[(3R)-3-amino-1-oxo-4-(2,4,5-trifluorophenyl)butyl]-5,6,7,8-tetrahydro-3-(trifluoromethyl)-1,2,4-triazolo[4,3-α]pyrazine (Chemical Formula 6a: Sitagliptin)

To a solution of 50.5 g (0.10 mole) of (R)-t-butyl 4-oxo-4-(3-(trifluoromethyl)-5,6-dihydro-[1,2,4]triazolo[4,3-α]pyrazin-7(8H)-yl)-1-(2,4,5-trifluorophenyl)butan-2-ylcarbamate, prepared in Example 4, in 150 ml of 2-propanol was slowly added 61 ml of conc. hydrochloric acid (35.0%), and the resulting mixture was stirred for 2 hrs or longer maintaining the temperature at 40° C. After monitoring the completion of the reaction by TLC, the reaction solution was cooled to room temperature, and slowly mixed with 4N NaOH to adjust the acidity into a pH of 6˜7. The reaction solution was concentrated, and 150 ml of dichloromethane was added to the concentrate. The acidity was adjusted into a pH of 12 by slowly adding 4 N NaOH and the reaction solution was extracted. The organic layers were pooled, washed with 150 ml of distilled water, dried over anhydrous magnesium sulfate, and concentrated under vacuum. The concentrate residue was crystallized in 150 ml of 2-propanol to afford 34.4 g (84.6%) of 7-[(3R)-3-amino-1-oxo-4-(2,4,5-trifluorophenyl)butyl]-5,6,7,8-tetrahydro-3-(trifluoromethyl)-1,2,4-triazolo[4,3-α]pyrazine (sitagliptin).

HPLC content: 99.8%

¹H NMR (CH₃OD, 400 MHz): 1.37 (s, 9H), 2.61-3.00 (m, 4H), 3.92-4.30 (m, 5H), 4.93 (s, 1H), 4.95-5.12 (m, 1H), 5.22-5.35 (br, 1H), 6.83-6.95, (m, 1H), 7.02-7.12 (m, 1H)

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

Using the novel intermediates of the present invention, highly pure DPP-IV inhibitors can be produced in a simple and economical manner at a high yield.

1. A compound, represented by the following Chemical Formula 1: 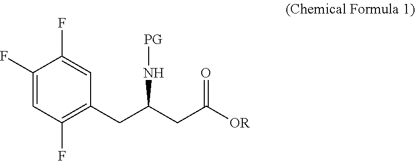
wherein,
R is pentafluorophenyl, 4-nitrophenyl, or 2-pyridyl, and
PG is an amine protecting group.

2. The compound according to claim 1, being selected from the group consisting of: (R)-pentafluorophenyl 3-(t-butoxycarbonylamino)-4-(2,4,5-trifluorophenyl)butanoate, represented by the following Chemical Formula 1a; 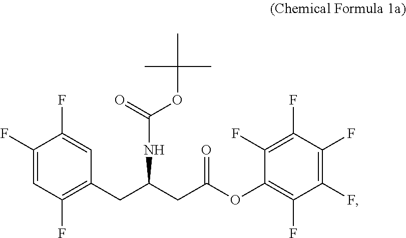 [SMILES: CC(C)(C)OC(=O)N[C@@H](CC(=O)Oc1c(F)c(F)c(F)c(F)c1F)Cc1cc(F)c(F)cc1F]
(R)-4-nitrophenyl 3-(t-butoxycarbonylamino)-4-(2,4,5-trifluorophenyl)butanoate, represented by the following Chemical Formula 1b; 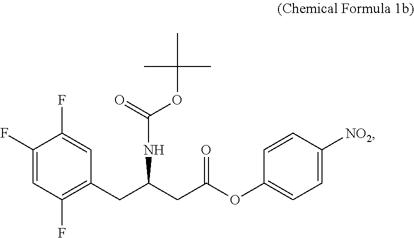 [SMILES: CC(C)(C)OC(=O)N[C@@H](CC(=O)Oc1ccc([N+](=O)[O-])cc1)Cc1cc(F)c(F)cc1F]
or
(R)-pyridin-2-yl 3-(t-butoxycarbonylamino)-4-(2,4,5-trifluorophenyl)butanoate, represented by the following Chemical Formula 1c

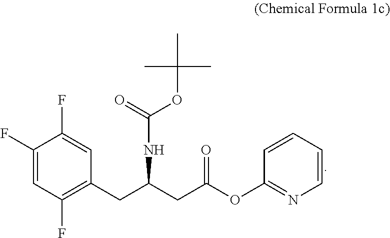 [SMILES: CC(C)(C)OC(=O)N[C@@H](CC(=O)Oc1ccccn1)Cc1cc(F)c(F)cc1F]

3. A method for preparing a compound represented by the following Chemical Formula 1, comprising reacting a compound represented by the following Chemical Formula 2 with a compound represented by the following Chemical Formula 3 in the presence of a base: 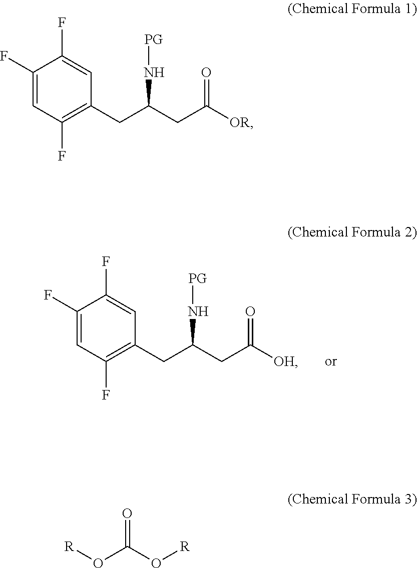
wherein,
R is pentafluorophenyl, 4-nitrophenyl, or 2-pyridyl, and
PG is an amine protecting group.

4. The method according to claim 3, wherein the respective compounds represented by the Chemical Formulas 2 and 3 are used at a molar equivalent ratio of 1:1 to 1:3.

5. The method according to claim 3, wherein the base is at least one selected from the group consisting of sodium carbonate, potassium carbonate, sodium hydroxide, potassium hydroxide, sodium bicarbonate, potassium bicarbonate, triethylamine, trimethylamine, pyridine, N-methylmorpholine, triisopropylamine and diisopropylethylamine.

6. The method according to claim 5, wherein the base is triethylamine.

7. The method according to claim 3, wherein the reaction is carried out in an organic solvent selected from the group consisting of 2-propanol, acetonitrile, ethylacetate, acetone, tetrahydrofuran, toluene, dichloromethane, dimethylacetamide, dimethylsulfoxide, dimethylformamide and a mixture thereof.

8. The method according to claim 7, wherein the organic solvent is dimethylformamide.

9. The method according to claim 3, wherein the reaction is carried out at a temperature of 0 to 80° C.

10. A method for preparing a DPP-IV inhibitor, comprising:
(S1) reacting a compound represented by the following Chemical Formula 2 with a compound represented by the following Chemical Formula 3 in the presence of a base to prepare a compound represented by the following Chemical Formula 1, using the method according to claim 3; and
(S2) reacting the compound represented by the Chemical Formula 1 with any one of compounds represented by the following Chemical Formulas 4a to 4c or a salt thereof to afford any one of compounds represented by the following Chemical Formulas 5a to 5c: 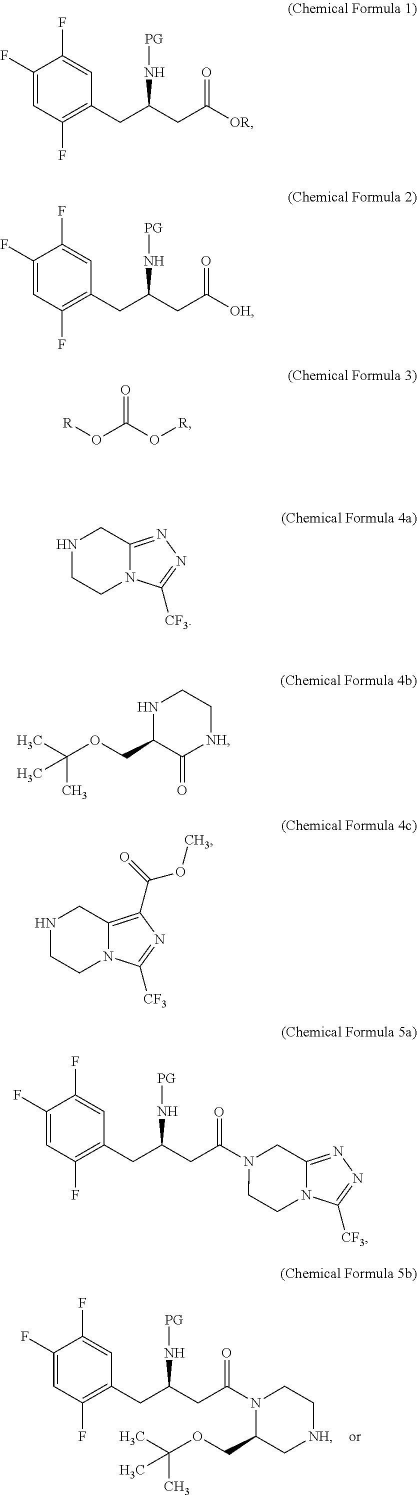 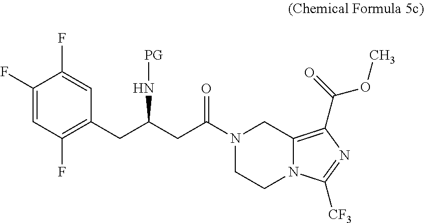
wherein,
R is pentafluorophenyl, 4-nitrophenyl, or 2-pyridyl, and
PG is an amine protecting group.

11. The method according to claim 10, wherein the step (S2) is carried out without isolating the compound of Chemical Formula 1, obtained in step (S1).

12. The method according to claim 10, wherein the compound of Chemical Formula 2 of step (S1), and the compound of any one of Chemical Formulas 4a to 4c or a salt thereof of step (S2), are used at a molar equivalent ratio of 1:1 to 1:3.

13. The method according to claim 10, wherein the reaction of step (S2) is carried out at a temperature of 0 to 80° C.

14. The method according to claim 10, further comprising (S3) removing the amine protecting group to afford any one of compounds represented by the following Chemical Formulas 6a to 6c: 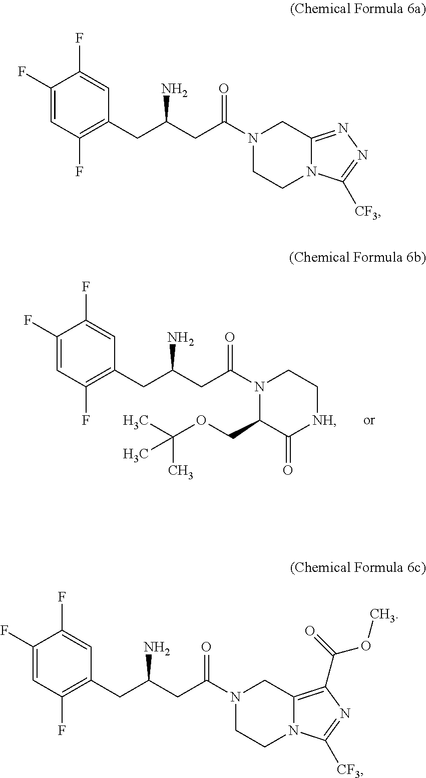 [SMILES: N[C@@H](CC(=O)N1CCn2c(nnc2C(F)(F)F)C1)Cc1cc(F)c(F)cc1F ; CC(C)(C)OC[C@@H]1C(=O)NCCN1C(=O)C[C@H](N)Cc1cc(F)c(F)cc1F ; COC(=O)c1nc(C(F)(F)F)n2c1CN(C(=O)C[C@H](N)Cc1cc(F)c(F)cc1F)CC2]
